(19) United States
(12) Patent Application Publication
(10) Pub. No.: US 2011/0125408 A1
(43) Pub. Date: May 26, 2011

(54) Thermoelectric biosensor for analytes in a gas

(76) Inventor: Lubna M. Ahmad, Chandler, AZ (US)

(21) Appl. No.: 12/002,901

(22) Filed: Dec. 18, 2007

Related U.S. Application Data
(63) Continuation of application No. 10/554,801, now abandoned.
(60) Provisional application No. 60/465,949, filed on Apr. 28, 2003.

(51) Int. Cl. G01N 33/48

(52) U.S. Cl. 702/19

(57) ABSTRACT

A biosensor for detecting at least one analyte in a gas includes a capture apparatus, a thermoelectrical sensor, and a microprocessor attached to the first ends of a first lead and a second lead, the two leads having second ends attached to the two contact pads of a thermopile whose difference is converted to concentration of the analyte and shown on an electronic display. The thermoelectrical sensor has a layer of at least one interactant, at least one thermopile and an insulator. The interactant with the analyte produces or consumes heat which is transferred to the thermopile which develops a voltage difference that causes the microprocessor to convert the voltage an indication of the analyte.

### TECHNICAL FIELD

The invention relates generally to an apparatus and method of detecting analytes in a gas and more specifically to a thermoelectrical biosensor for measuring acetone in expelled air for monitoring ketone production, for example, in diabetes or weight loss.

### BACKGROUND

When the cellular concentrations of glucose are low, the body begins to metabolize fat to produce energy. Ketones are the byproducts of this fat metabolism. In particular, three ketone bodies are formed: β-hydroxybutyrate (β-HBA), acetoacetate, and acetone. In diabetics, when insulin levels are low and blood glucose levels are high, often large amounts of ketones are released into the bloodstream. This could potentially cause diabetic ketoacidosis (DKA).

Patients in DKA typically experience nausea, fatigue, rapid breathing and emit a fruity odor in their breath. To clinicians and other trained personnel, this fruity odor is distinct and attributable to acetone, which is a volatile ketone body released into alveolar air. The concentration of alveolar acetone is an accurate measure and description of ketone bodies in the bloodstream.

Diabetics are strongly cautioned about conditions such as DKA. Untreated DKA can result in catalepsy or even death. However, DKA is very preventable if ketone levels are monitored and high ketone counts are immediately reported to medical personnel. The current methods of ketone measurement are blood and urine analysis. The current blood tests are accurate; however, their invasive nature is undesirable and frequently causes patients to delay treatment. Blood tests also are expensive, as a number of products are needed, including a lancet for blood letting, test strips, a specialized device and batteries. Urine analysis, as several studies have shown, is not a good representation of the body's current blood ketone level as it simply monitors stagnant urine from the bladder. The epidemic of diabetes in the United States will contribute to staggering medical costs, which can be limited by close ketone monitoring and maintenance.

Ketone monitoring also is becoming recognized as a tool for nutritionists to monitor fat burning during dieting. Individuals who are dieting take in fewer calories than required. The other required calories are obtained from body metabolism of fat. Several studies show that breath acetone concentrations are good representations of fat burning during a calorie deficit. A direct correlation between breath acetone and average fat loss has been established. Obesity has become increasingly prevalent and has now reached epidemic levels and is consequently of great concern to healthcare professionals. Much effort has been invested in treating obesity and promoting healthy weight loss programs for obese individuals. The traditional weight-scale reading does not accurately reflect fat loss, as weight also varies with water loss/gain, muscle development, and other factors. For treatment of obesity, a sensor measuring fat burning is needed to adjust weight management plans to individual physiology. A non-invasive, inexpensive, simple-to-use acetone sensor would be an appropriate tool for nutritionists, dietitians and obese individuals seeking to monitor fat metabolism.

Some systems for measuring analytes in air operate on electrochemical principles (U.S. Pat. No. 5,571,395, issued Nov. 5, 1996, to Goldstar Co., Ltd.) and infrared detection (U.S. Pat. No. 4,391,777 issued Jul. 5, 1983, to Cal Detect, Inc.). U.S. Pat. No. 6,658,915, issued Dec. 9, 2003, to Cyrano Sciences, Inc. has chemically sensitive resistors to detect airborne substances and requires the use of an electrical source.

What is needed is a faster, inexpensive, non-invasive method of measuring analytes in fluids, particularly gas, that does not require power.

### SUMMARY OF THE INVENTION

In one embodiment, there is a biosensor for detecting at least one analyte in a gas. The biosensor includes a capture apparatus, a thermoelectric sensor, and a microprocessor. The sensor has a layer of at least one analyte interactant and at least one thermopile. The microprocessor is attached to first ends of a first lead and a second lead, the first lead having a second end attached to one of the thermopile contact pads and the second lead having a second end attached to the other thermopile contact pad. The passage of gas containing the analyte through the capture apparatus brings the analyte into contact with the interactant that produces or consumes heat transmitted to the thermopile that then registers a voltage difference in the microprocessor, which converts the voltage and indicates the presence of the analyte.

Optionally, the interactant is selected from a chemical reactant, catalyst, adsorbent, absorbent, vaporization agent or a combination thereof.

In another embodiment, the sensor can have multiple thermopiles, each having the same or different interactants, which are each independently connected to the microprocessor via two leads, thereby providing a display of single or multiple analytes.

In one embodiment, the interactant is selected from sodium hypochlorite, salt, sodium dichloroisocyanurate, nitrosyl chloride, chloroform or a combination thereof.

In another embodiment the biosensor's interactant is selected from sodium hypochlorite, sodium dichloroisocyanurate, salt, nitrosyl chloride, chloroform or a combination thereof.

In another embodiment, the biosensor's microprocessor also communicates with an electronic display, noise maker, other output or a combination thereof.

In another embodiment, the analyte is acetone, whose presence indicates the presence of ketones in the bloodstream.

In another embodiment, the interactant is specific for ethanol and/or alkanes, whose presence may indicate various pathologies, such as breast cancer and transplant rejection.

In other embodiments, interactant(s) can be specific for airborne environmental toxins or classes of toxins, chemical warfare agents, biological warfare agents, and other airborne components of gas mixtures.

In another embodiment, the thermopile is fabricated from bismuth/antimony, other metals, alloys, semiconductor materials, or liquid thermoelectric materials.

In yet another embodiment the analyte interactant comprises biologically active materials comprising cells, cell organelles, micro-organisms or genetically modified organisms.

In another embodiment, compounds present in the air stream other than the analyte of interest facilitate the production or consumption of heat.

In another embodiment, the gas stream is replaced by a liquid.

In another embodiment, there is a method of detecting an airborne analyte by thermoelectric sensor. The method includes providing a thermoelectric sensor, the sensor having a layer of at least one analyte interactant that when combined with the analyte gives off or consumes heat, at least one thermopile to which the heat change is transferred, which then registers a voltage difference; a microprocessor attached to first ends of a first lead and a second lead, the first lead having a second end attached to one of the contact pads of the thermopile and the second lead having a second end attached to the second contact pad of the thermopile, the microprocessor also communicating with a signal device. The next steps include passing an air stream containing the airborne analyte over the thermoelectric sensor, and indicating the presence of the airborne analyte.

The step of providing the thermoelectric sensor can also include providing an analyte interactant specific for acetone and the provided display indicates the presence of acetone, whereby the burning of fat is indicated.

In another embodiment, the provided thermoelectric sensor has multiple thermopiles, each having the same or different analyte interactants and each being connected to the microprocessor by two leads, and the presence of one or multiple airborne analytes are indicated.

In another embodiment, indicating the presence of the airborne analyte also includes indicating the concentration of the analyte.

In still another embodiment, there is a biosensor for detecting at least one ketone in expired air and the occurrence of a fat-burning state. This biosensor has a capture apparatus, a thermoelectric sensor, a microprocessor and a display. The sensor includes a layer of at least one interactant specific for the ketone and at least one thermopile having a first and a second contact pad. The microprocessor is attached to first ends of a first lead and a second lead, the first lead having a second end attached to the first thermopile contact pad and the second lead having a second end attached to the second thermopile contact pad. The display is connected to the microprocessor for indicating the presence or quantity of at least one ketone.

In another embodiment, the interactant is selected from a chemical reactant, catalyst, adsorbent, absorbent, vaporization agent or a combination thereof. Optionally, the biosensor has multiple thermopiles, which each are in contact with the same or different interactants, which are each independently connected to the microprocessor via two leads. The interactant is selected from sodium hypochlorite, sodium dichloroisocyanurate, salt, nitrosyl chloride, chloroform or a combination thereof. The microprocessor also communicates with an electronic display, noise maker, other output or a combination thereof. The analyte can be acetone, whose presence indicates the presence of ketones in the bloodstream. The thermopile can utilize bismuth/antimony, other metals, alloys, semiconductor materials, or liquid thermoelectric materials. The analyte interactant can be biologically active materials comprising cells, cell organelles, micro-organisms or genetically modified organisms. Optionally, the biosensor has at least one compound present in the air stream, other than the analyte of interest, that facilitates the production or consumption of heat.

In yet another embodiment, a biosensor for detecting at least one volatile environmental toxin or class of toxins and chemical and biological warfare agents in air is disclosed. The biosensor has a thermoelectric sensor, a means for transmitting the voltage change from at least one thermopile to a microprocessor, a microprocessor capable of processing the voltage change(s) from the at least one thermopile, a means for transmitting the processed information from the microprocessor to a signal output device, and a signal output device. The sensor has a layer of at least one interactant specific for the agent and at least one thermopile having a first and a second contact pad between which a voltage change is capable of developing.

In other embodiments, the microprocessor further is preprogrammed for a location of at least one thermopile and for a correlation of the voltage change with the location. The interactant is selected from a chemical reactant, catalyst, adsorbent, absorbent, vaporization agent or a combination thereof. Multiple thermopiles, each having the same or a different interactant, each independently transmit voltages to the microprocessor. The signal output device can be an electronic display, noise maker, other output or a combination thereof. The thermopile can be fabricated from bismuth/antimony, other metals, alloys, semiconductor materials, or liquid thermoelectric materials. The analyte interactant can be biologically active materials such as cells, cell organelles, micro-organisms or genetically modified organisms. Optionally, compounds present in the air stream other than the analyte of interest facilitate the production or consumption of heat.

### BRIEF DESCRIPTION OF THE DRAWINGS

FIG. 1 is a schematic showing a rectangular thermopile.

FIG. 2 is a schematic showing a circular thermopile.

FIG. 3 shows an individual blowing into the biosensor, on which there is an electronic display. Below is an enlarged schematic of the biosensor; arrows show the direction of expired air flow therein; and there is an enlarged thermoelectric sensor therein.

FIG. 4 is a graph showing the response of a bare thermopile to a flowing air stream, with and without 1% acetone.

FIG. 5 is a graph showing the response of a thermopile coated with a chemical reagent to a flowing air stream with and without 1% acetone.

FIG. 6 shows FIG. 4 overlaid with FIG. 5. Clearly the variations in current are entirely due to the chemical and heat imparted to the thermopile.

FIG. 7 is a schematic of the sensor for use in a fluid.

FIG. 8 is a schematic of the sensor for use in a fluid.

### BEST MODES FOR CARRYING OUT THE INVENTION

A measuring device is required to assess the concentrations of biochemical or chemical substances expired by the body or contained within other fluid mixtures. Most biosensors and chemical sensors have detected analytes only in fluids. The instant invention discloses a thermo-electric air analyzer, which is a self-contained unit that advantageously does not require a power source. It is noninvasive and provides a representation of analytes in the bloodstream but does not access with the bloodstream.

More that 200 analytes have been identified in human breath. Examples include but are not limited to pentane (and other members of the alkane family), isoprene, benzene, acetone (and other members of the ketone family), alcohols (ethanol, methanol, isopropanol, etc), ammonia (particularly in uremia and kidney failure), reflux, medication (particularly at high levels), and substances which interfere with common alcohol detection systems (e.g., acetone, acetaldehyde, acetonitrile, methylene chloride, methyl ethyl ketone, toluene, etc.) The analyte need not be vaporized from the bloodstream into the alveoli to be detected; non-vaporized substances carried in water vapor also can be detected.

In operation, the biosensor receives gas or breath containing an unknown amount of an analyte, for example, acetone. Air passes over an interactant such as a co-reactant, catalyst absorbent with which the acetone interacts. The interaction is either exothermic or endothermic, thereby producing a temperature gradient over the highly conductive metal of the thermopile. More than one interaction can also occur simultaneously and increase the amount of heat that is generated or consumed. Multiple types of interactants can be deposited onto a single surface or sequential reactions can occur thereon. For analyte interactions, it is desirable for the analyte to be the rate-limiting reagent.

The generated heat induces a voltage difference between the two contact pads of the thermopile. The measured voltage is proportional to the heat generated or consumed by the analyte interactions, which in turn is related to the amount of the analyte.

When a temperature gradient is produced in a conducting material such as metal, electrons tend to travel toward the colder region. This movement causes a potential difference between the two sides. The voltage across the device is proportional to the temperature gradient that instigated the electron motion according to the Seebeck effect: V₁₂=S₁₂(T₂−T₁). The voltage is proportional to three things: (a) the temperature difference, T₂−T₁, (b) the number of thermocouples attached in series, and (c) the EMF of the two metals or the Seebeck coefficient. EMF=n*s*(t₂−t₁), where n=number of thermopiles. The two metals with the greatest thermoelectric EMF are antimony and bismuth.

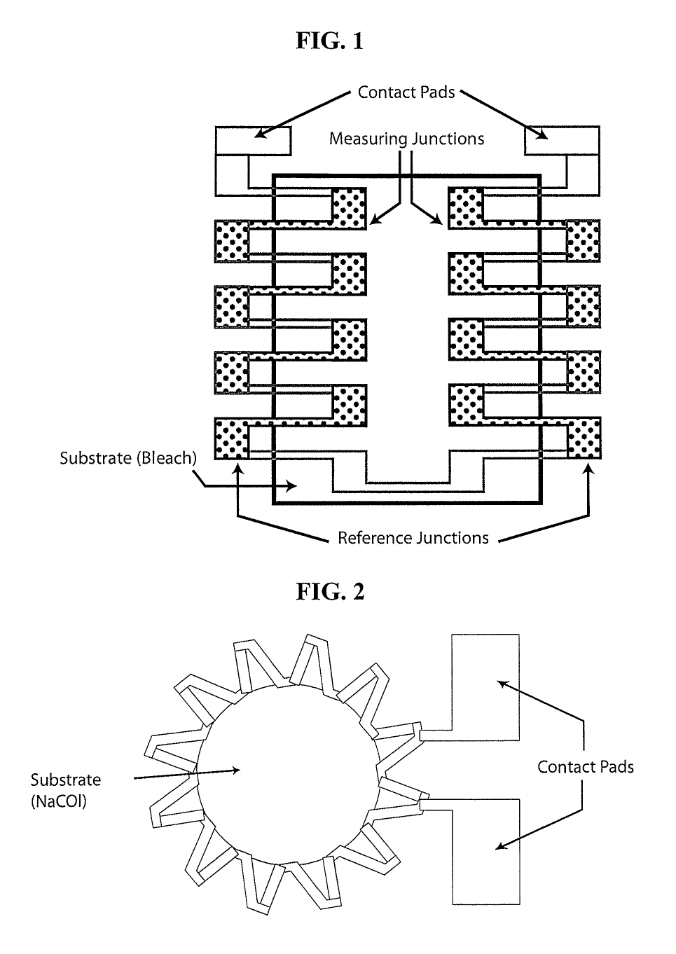

The sensor of choice for measuring this effect is a thermocouple, particularly thermocouples connected in series to form a thermopile. Two general forms of thermopiles are shown schematically in FIGS. 1 and 2. The exact geometry can vary, as can the metals selected for the particular thermopile. Two or more metals can be joined at a thermoelectric junction. As an alternative to pure metals, the thermopile can be constructed with alloys, semiconductor materials, liquid thermoelectric materials or other materials with or without dopants commonly used to construct thermopiles. The invention is not limited by the materials used in the thermopile. In the instant invention the heat produced or consumed affects the sensing junctions but does not directly affect the reference junctions, allowing for the potential differential to be produced. Alternatively, the chemical reactant or the adsorbent can be placed over the reference junctions. In this case the heat produced does not directly affect the measuring junctions. In both of these configurations, the voltage is measured between two contact pads.

In addition, multiple thermopiles may be linked in arrays. Several thermopiles can have the same interactant to detect the same analyte; their voltages could be averaged by a microprocessor with the result that the effect of noise would be easier to reduce. Several thermopiles may detect a single analyte with different interactants. In other cases, each thermopile within the array may be coated with a different material such that selectivity of several analytes is determined by the different reactants/adsorbents. From the voltage of individual thermopiles within the array, a fingerprint pattern for each analyte or combination of analytes may be determined. One example of the latter is the provision of interactants specific for ethanol and alkanes, such that fingerprints for various pathologies are revealed. Such pathologies include breast cancer and transplant rejection.

The heat which is generated by the presence of the analyte may come from the analyte interaction in a variety of ways. The analyte-interactant produces heat by any of a variety of ways, including but not limited to chemical reaction, adsorption, absorption, vaporization, a combination thereof or any other way that generates heat when the analyte contacts the interactant. Biochemical reactions such as DNA and RNA hybridization, protein interaction, antibody-antigen reactions also can be used to generate or diminish heat in this system. The analyte can interact not only with chemicals but also with materials from living systems or living systems themselves. Examples include but are not limited to microorganisms, cell, cellular organelles and genetically modified versions thereof. Chemicals, including but not limited to, environmental toxins, chemical warfare agents and biological warfare agents, can kill cells or impair organelle function, thus reducing heat in the system. The interaction of the analyte can also involve interaction with other substances in the air, including but not limited to oxygen, nitrogen, carbon dioxide and water.

Other biosensors can detect other analytes. For example, alcohol can interact with a chemical such as chromium trioxide (CrO₃) or enzymes such as alcohol dehydrogenase, alcohol oxidase, acetoalcohol oxidase.

Interactants can be adsorbents including but not limited to activated carbon, silica gel, and platinum black. Suitable chemical reagents include but are not limited to halogen compounds (HCl and Cl₂), activated carbon with halogenated compounds, sodium hypochlorite, calcium hypochlorite, sodium monochloro-s-triazimethione, sodium dichloro-s-triazimethione sodium trichloro-s-triazimethione, and chromium trioxide. Suitable hydrogenation reagents include Raney nickel and platinum catalysts.

When environmental chemicals are to be detected, the thermoelectric sensor may be part of a telemetry system. With the thermoelectric sensor there may be an amplifier to transmit signals such as radiowaves to a microprocessor. The amplifier, unlike the thermoelectric sensor, may need a power source, such as a battery or solar-powered cell.

A microprocessor is connected to the thermopile by two leads or other transmission system as in the above-mentioned telemetry. Microprocessors are well known in the art and can be readily selected by those of ordinary skill in the art. The microprocessor is programmed to convert the electrical or other signals to representations of the analyte presence, quantity or other output. As needed, the microprocessor can be programmed to assess the location of one of multiple sensors in a telemetry system or the combination of signals of multiple sensors for the same or different analytes. The microprocessor can be programmed to form a control loop or provide feedback. In other cases, the microprocessor analyzes data and transmits commands to operate a drug delivery device or other type of medical device. In most cases, the microprocessor transmits a signal to an indicator. The indicator can be an integral part of the microprocessor or separate.

The indicator can be a display showing the presence of the analyte, a level of the analyte, or a diagnosis, when an array of thermopiles sends multiple messages for processing at the microprocessor. For example, when ketones are the analyte, there can be colored indicators for the seriousness of the diabetic ketoacidosis or for the success of fat burning. The indicator also may be an alarm, particularly for environmental toxins or biowarfare hazards. The indicator can be a combination of any of the above.

The inventive biosensor can be used in microfluidic devices for various applications including but not limited to biochemical analysis, drug testing, blood chemistry analysis, medical diagnosis, forensics and pharmaceutical screening. Microfluidic devices may be used to analyze both liquids and gases. Microfluidic devices have gained significant interest recently due to their ability to perform multiple processes in very short time intervals and in very little space. The inventive thermopile-based sensors are ideally suited for use in microfluidic devices as a sensing modality to detect or measure analytes. The fact that no power needs to be supplied to the thermopile is particularly advantageous in these applications.

### EXAMPLES

### Example 1

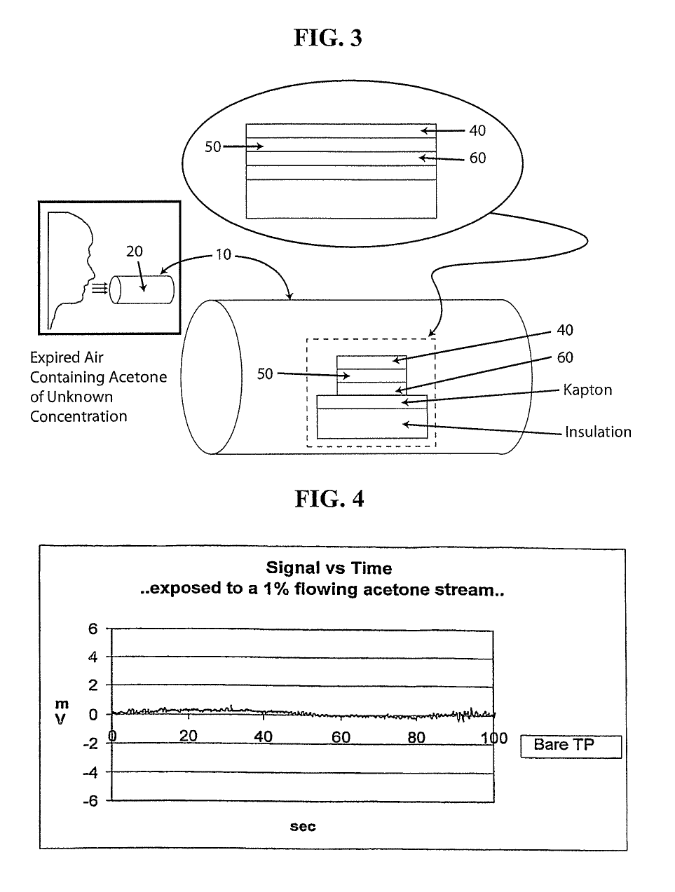

FIG. 3 shows a general system of a thermoelectric biosensor 10. The middle part of the figure shows an individual blowing into a capture tube 20 from which a display (not shown) projects. The expelled air flows over a layer 40 of interactant (e.g., chemicals, catalysts and/or absorbents) which interact with ketone in the air or gas mixture. As the reactants form or the acetone is absorbed, heat is given off or consumed, and the resulting temperature change is converted by the thermopile layers 50 and 60 to a voltage difference. A microprocessor with display has dual leads, one lead to one of the thermopile contact pads and the other lead to the second thermopile contact pad, which register the difference in voltage between the two thermopile contact pads and converts the voltage difference to acetone content in parts per million (PPM) or other convenient signals.

For interaction with ketones, any reactant, catalyst, enzyme or adsorbent that produces significant heat or consumes significant heat when exposed to acetone is a viable candidate for immobilization to the active junctions (either measuring or reference) of the thermopile. Likely candidates are sodium hypochlorite (NaOCl), sodium dichloroisocyanurate (also known as sodium troclosene and sodium dichloro-s-triazinetrione), sodium monochloro-s-triazimethione, sodium trichloro-s-triazimethione, calcium hypochlorite (Ca(OCl)₂), salt (NaCl), nitrosyl chloride (ClNO), and chloroform (CHCl₃) in the presence of base.

As products of the first interaction form, the products can potentially be used as co-reactants for the initiation of secondary interactions, which could “amplify” temperature changes and the thermoelectric effect.

The capture tube can be made of any firm material, such as a metal or plastic, which will not interfere with the heat generating or dissipating process. Although the shape in FIG. 3 is that of a cylinder, the capture tube may be made in other geometries including, for example, a cuboidal geometry. It may also have a narrow mouthpiece at one end. The main tube can be made for reuse, and the mouthpiece can be detachable and replaceable. Alternately the capture tube can be as narrow as a mouthpiece. The inside of the capture tube is preferably shaped to allow the entering air to flow in a laminar fashion over the surface of the thermopile and its immobilized interactant. Laminar flow is especially desired in this case, as it minimizes the background thermal noise, contributing to a larger signal to noise ratio. The laminar flow may or may not be fully developed at the thermopile. With some analytes and fluids, the passage of analyte fluid over the thermopile may be turbulent. For environmental toxins where the biosensor needs to be exposed to the ambient air, the capture tube may only consist of an overlying shelter to protect the sensor from the elements, particularly dust.

In reducing the invention to practice, a thermopile design with 50 thermopile junctions is employed using the configuration shown in FIG. 1. At a minimum, there must be at least two junctions. The maximum number of junctions is limited by the increasing thermopile electrical resistance with greater numbers of junctions. The materials in the two thermopile layers are different and can be chosen from a variety of metals, alloys, semiconductor materials, liquid thermoelectric materials, or other materials with or without dopants commonly used to construct thermopiles. A preferred metal combination is antimony-bismuth. Sb—Bi films have particularly high thermal thermoelectric EMF. The thickness of the metal layers is typically small. The thermopile metal layers can be formed by sequentially evaporating two metals onto a substrate. One side of the substrate is in contact with the first metal deposited. The other side of the substrate may be placed in contact with insulation to minimize heat loss. The normally very thin substrate, consisting of, for example, materials like MYLAR® DuPont polyester film or KAPTON® DuPont polyimide film, is chosen for its strength and low thermal conductivity.

### Example 2

The above disclosed biosensor apparatus was tested as follows. Before the chemical layer was added atop the thermopile, the thermopile was subjected to an air mixture containing 1% acetone or no acetone. The results are shown in FIG. 4. The sensor output signal was recorded as very small voltage fluctuations that did not appear to vary between acetone (shown as “Actn” on the graph) and air alone.

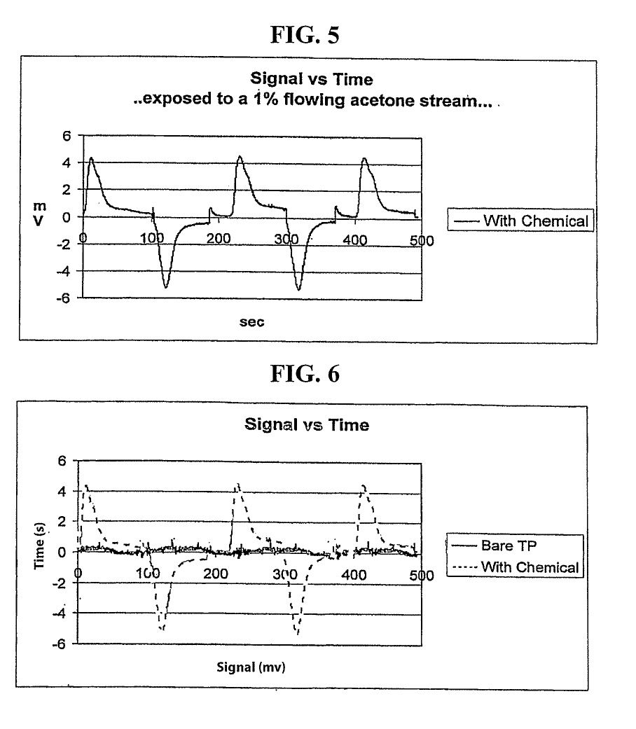

FIG. 5. shows the results after the chemical reagent was applied to the thermopile. The positive peaks coincided with the application of air with 1% acetone, indicating an effective exothermic reaction. The negative peaks coincided with exposure of the biosensor to air without acetone and gradually diminished to “0”. It is believed that the negative peaks are due to the reverse endothermic reaction.

FIG. 6 shows the overlap of FIGS. 4 and 5. Clearly, when the acetone is exposed to the thermopile immobilized with a reagent, a significant signal is detected. The differences between graphs of the thermopile response with and without chemical layer indicate that the large swings in voltage are due entirely to the interaction of acetone with the chemical layer.

### Example 3

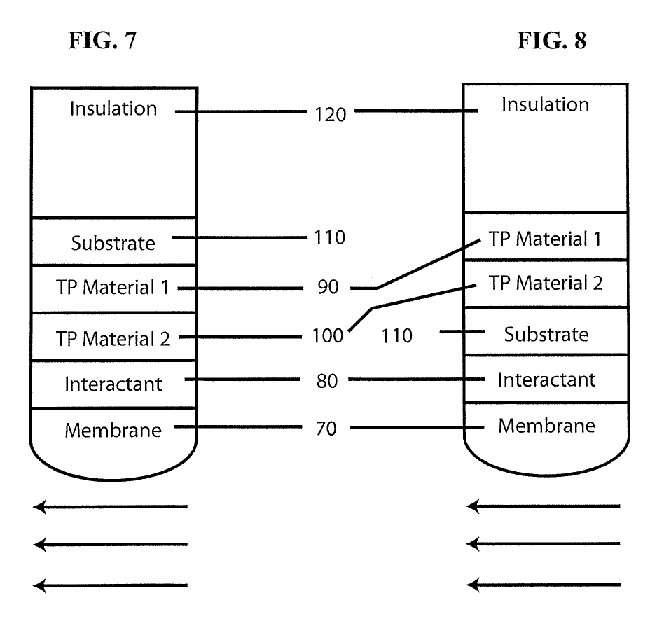

FIGS. 7 and 8 show two other possible arrangements of the thermoelectric sensor for use in a gas or liquid. Note that the sensor is covered with membrane 70 to limit interaction to that specifically designed for and to limit corrosion by the fluid. Under the membrane are the interactant 80, thermopile materials 1 and 2 (90 and 100, respectively), substrate 110 and insulation 120. The sensor can be insulated to limit direct transfer of the liquid temperature to the thermopile, thus increasing the sensitivity of the thermopile to the temperature change of the interactant-analyte.

It should be appreciated from the foregoing description that the present invention provides an improved vapor sensing instrument that is sufficiently small and lightweight to be handheld. Other uses of a breath biosensor include monitoring the breath for chemical and biochemical compounds of interest, including but not limited to ethanol and alkanes, which may indicate various pathologies, such as breast cancer and transplant rejection. The biosensor chemical layer can be varied to react with specific volatile and/or airborne environmental toxins or classes of toxins, chemical warfare agents, biological warfare agents, and other airborne or components of gas mixtures that might warrant detection.

Although the invention has been described in detail with reference to the presently preferred embodiments, those of ordinary skill in the art will appreciate that various modifications can be made without departing from the invention. Accordingly, the invention is defined only by the following claims.

1. A biosensor for detecting at least one analyte in a gas, the biosensor comprising:
a) a capture apparatus; and
b) a thermoelectric sensor, the sensor comprising a layer of at least one analyte interactant that increases or decreases in temperature and at least one thermopile, said at least one thermopile having a first contact pad and a second contact pad;
wherein the passage of gas containing the analyte through the capture apparatus brings the analyte into contact with the interactant and produces or consumes heat, which is transmitted to the thermopile that then produces a voltage difference and measures the analyte.

2. The biosensor of claim 1, wherein the interactant is selected from a chemical reactant, catalyst, adsorbent, absorbent, vaporization agent or a combination thereof.

3. The biosensor of claim 1, wherein the biosensor further comprises multiple thermopiles, each having the same or different interactants.

4. The biosensor of claim 1, wherein the interactant is selected from sodium hypochlorite, sodium monochloroisocyanurate, sodium trichloroisocyanurate, salt, nitrosyl chloride, chloroform or a combination thereof.

5. The biosensor of claim 36, wherein the microprocessor also communicates with an electronic display, noise maker, other output or a combination thereof.

6. The biosensor of claim 1, wherein the analyte is acetone, whose presence indicates the presence of ketones in the bloodstream.

7. The biosensor of claim 1, wherein the analyte is alcohol and the interactant is selected from chromium trioxide, alcohol dehydrogenase, alcohol oxidase, acetoalcohol oxidase or a combination thereof.

8. The biosensor of claim 1, wherein the interactants are specific for ethanol and alkanes, thereby indicating various pathologies, such as breast cancer and transplant rejection.

9. The biosensor of claim 1, wherein the interactant is specific for airborne environmental toxins or classes of toxins, and other airborne components of gas mixtures, thereby detecting these compounds.

10. The biosensor of claim 1, wherein the thermopile comprises metals, alloys, semiconductor materials, or liquid thermoelectric materials.

11. The biosensor of claim 1, wherein the analyte interactant comprises biologically active materials comprising cells, cell organelles, micro-organisms or genetically modified organisms.

12. The biosensor of claim 1, wherein compounds present in the gas other than the analyte of interest facilitate the production or consumption of heat.

13. (canceled)

14. A method of detecting an analyte in a gas by thermoelectric sensor, the method comprising
a) providing a thermoelectric sensor, the sensor comprising
i) a layer of at least one analyte interactant that when combined with the analyte, gives off or consumes heat; and
ii) at least one thermopile to which the heat change is transferred, which then registers a voltage difference, the at least one thermopile having a first contact pad and a second contact pad;
b) passing a gas containing the airborne analyte over the thermoelectric sensor; and
c) indicating the presence or quantity of the analyte.

15. The method of claim 14, wherein the step a of providing the thermoelectric sensor comprises providing an analyte interactant specific for acetone and step c comprises a display indicating the presence of acetone, whereby lipid metabolism is indicated.

16. The method of claim 14, wherein the provided thermoelectric sensor has multiple thermopiles, each having the same or different analyte interactants, whereby the presence of single or multiple analytes are indicated.

17. The method of claim 14, wherein step c also comprises indicating the concentration of the analyte.

18-35. (canceled)

36. The biosensor of claim 1, wherein the biosensor further comprises
c) a microprocessor attached to first ends of a first lead and a second lead, the first lead having a second end attached to said first thermopile contact pad and the second lead having a second end attached to said second thermopile contact pad,
whereby the voltage difference produced by the thermopile is registered in the microprocessor, which converts the voltage so as to measure the analyte.

37. The biosensor of claim 36, wherein multiple thermopiles, each having the same or different interactants, are each independently connected to the microprocessor via two leads.

38. The method of claim 14, wherein the thermoelectric sensor provided in step a. further comprises
iii) a microprocessor attached to first ends of a first lead and a second lead, the first lead having a second end attached to the first thermopile contact pad and the second lead having a second end attached to the second thermopile contact pad, the microprocessor also communicating with a signal device.

39. The method of claim 38, wherein the provided thermoelectric sensor has multiple thermopiles, each having the same or different analyte interactants and each being connected to the microprocessor by two leads, whereby the presence of single or multiple analytes are indicated.
